(19) United States
(12) Patent Application Publication
(10) Pub. No.: US 2006/0100459 A1
(43) Pub. Date: May 11, 2006

(54) Method for preparing an unsaturated carboxylic acid

(76) Inventor: Roland Jacquot, Francheville (FR)

Correspondence Address: Jean-Louis Seugnet;Rhodia, 259 Prospect Plains Road, CN 7500, Cranbury, NJ, 08512, US

(21) Appl. No.: 10/518,775

(22) PCT Filed: Jun. 18, 2003
(86) PCT No.: PCT/FR03/01856

§ 371 Date: Aug. 11, 2005

(30) Foreign Application Priority Data
Jun. 21, 2002 (FR) 02/07731

(51) Int. Cl. C07C 51/235

(52) U.S. Cl. 562/408; 562/535

(57) ABSTRACT

The invention concerns a method for preparing an unsaturated carboxylic acid from the corresponding aldehyde. More particularly, the invention aims at preparing an aliphatic carboxylic acid having at least an unsaturation conjugated with the carbonyl group. In particular, the invention concerns the preparation of geranic acid. The invention relates to a method for preparing an unsaturated carboxylic acid from the corresponding aldehyde. Said method is characterized in that it comprises a step which consists in oxidizing said aldehyde, in controlled basic medium and using molecular oxygen or a gas containing same, in the presence of a catalyst based on palladium and/or platinum and an activator based on bismuth, in conditions such that the oxidizing is carried out by diffusion process

The subject of the present invention is a method for preparing an unsaturated carboxylic acid from the corresponding aldehyde.

More particularly, the invention aims at preparing an aliphatic carboxylic acid having at least one unsaturation conjugated with the carbonyl group.

It relates in particular to the preparation of geranic acid.

The preparation of an unsaturated carboxylic acid, in particular of geranic acid, is not easy to carry out.

Most of the methods described in the literature involve a chemical oxidant. Thus, the preparation of geranic acid by oxidation of citral, in the presence of silver nitrate and in a basic medium, has been described [J. Prakt. Chem. (1936), 147, 199]. Such a method involves an expensive reagent used in a large quantity.

It is also known to carry out the oxidation with manganese oxide used in a large excess [Corey et al., J.A.C.S, (1968), 90, 5616] or with sodium chlorite [Dalcanale et al., J. Org. Chem., 51(4), 567-9].

None of the methods described for the oxidation of citral involves a catalytic system, but involves an oxidant used in a stoichiometric quantity.

Moreover, the preparation of an unsaturated aliphatic carboxylic acid by oxidation of an alcohol, namely 9-decen-1-ol, with air, in a dioxane-water medium, in the presence of supported palladium or platinum catalysts and potassium hydroxide, has been described [A.-B. Crozon et al., New. J, Chem., (1998), 269-273]. It is possible to obtain 9-decenoic acid from 9-decen-1-ol.

However, while the method described is applied to carry out the oxidation of citral, the applicant has observed that it was not possible to obtain geranic acid but only a ketone, 6-methyl-4-hepten-2-one.

The objective of the present invention is to provide a method involving a catalytic system.

There has now been found, and that is what constitutes the subject of the present invention, a method for preparing an unsaturated carboxylic acid from the corresponding aldehyde, which comprises a step for oxidizing said aldehyde, in a controlled basic medium and using molecular oxygen or a gas containing it, in the presence of a catalyst based on palladium and/or platinum and of an activator based on bismuth, under conditions such that the oxidation occurs in a diffusion mode.

It has been found in particular that an unsaturated aliphatic carboxylic acid could be obtained by oxidizing a compound comprising one or two double bonds insofar as both the reaction mode which should be a diffusion mode and the basicity of the medium would be controlled.

In the present text, the expression “diffusion mode” also called “physical mode” is understood to mean a mode which corresponds to the conventional definition known to persons skilled in the art.

To this effect, reference may be made to the various manuals by J. RICHARDSON, Principles of catalyst development (1989), Plenum Press New York and by J. VILLERMAUX, Genie de la reaction chimique: conception et fonctionnement des réacteurs [Chemical reaction technology: reactor design and operation] (1993), Lavoisier.

Diffusion mode conditions are conditions such that the dissolved oxygen concentration in the medium is close to zero.

The method of the invention applies most particularly to the oxidation of all aldehyde-type compounds which are likely to undergo degradation or isomerization during the oxidation reaction in a basic aqueous medium.

The aldehyde is therefore an aliphatic or cycloaliphatic aldehyde having at least one unsaturation, a double bond or a triple bond.

The method of the invention applies fully to an aliphatic aldehyde having two double bonds of which at least one is conjugated with the carbonyl group.

The invention preferably relates to the use of a terpene-type unsaturated aldehyde. The expression “terpene” is understood to mean oligomers derived from isoprene. Said substrate comprises a multiple number of carbon atoms of 5. The expression “total number of carbon atoms” includes the formyl group.

The aldehyde used in the method of the invention may be symbolized by the following formula:
A-CHO (I)
in said formula (I):

A represents a hydrocarbon group having at least one unsaturation having from 4 to 19 carbon atoms which may be a linear or branched, saturated or unsaturated acyclic aliphatic group; a monocyclic or polycyclic, saturated or unsaturated or aromatic carbocyclic group; a linkage of a saturated or unsaturated aliphatic group and/or of a saturated, unsaturated or aromatic carbocycle.

Thus, the unsaturation may be carried by an aliphatic hydrocarbon chain and/or alternatively contained in a ring.

The substrate used in the method of the invention corresponds more particularly to formula (I) in which A represents an unsaturated acyclic aliphatic group.

A represents a linear or branched acyclic aliphatic group preferably having from 4 to 19 carbon atoms comprising one or more unsaturations in the chain, generally 1 to 5 unsaturations which may be single or conjugated double bonds or triple bonds: it being possible for the unsaturation to be at the chain end and/or alternatively inside the chain and/or conjugated with the CO group.

The hydrocarbon chain may optionally carry one or more substituents insofar as they do not react under the reaction conditions and there may be mentioned in particular a halogen atom or a trifluoromethyl group.

The preferred unsaturated aliphatic substrates are those which correspond to formula (I) in which A represents a linear or branched alkyl group having from 4 to 19 carbon atoms and comprising at least one double bond, preferably two double bonds of which at least one is conjugated with the CO group.

As more specific examples of A, there may be mentioned the groups comprising 8 carbon atoms having a double bond, and bearing two methyl groups, preferably at the 3- and 7-position.

By way of examples of A, there may be mentioned an octen-7-yl group, a 2,6-dimethylheptadi-1,5-nyl group.

In the compounds corresponding to formula (I), A may represent a cyclic aliphatic group containing a double bond in the ring.

A represents a carbocycle having from 3 to 8 carbon atoms in the ring, preferably 5 or 6 and comprising 1 or 2 unsaturations in the ring, preferably 1 or 2 double bonds. In this case, the double bond is contained in the ring.

The preferred unsaturated cycloaliphatic substrates are those which correspond to formula (I) in which A represents a cycloalkyl group having from 3 to 8 carbon atoms, preferably 5 or 6 and comprising a double bond.

As preferred examples of A groups, there may be mentioned in particular cyclopentene, cyclohexene, 1-methylcyclohex-1-ene, 4-methylcyclohex-1-ene, cycloheptene and menthene.

A represents a polycyclic carbocyclic group comprising from 3 to 6 carbon atoms in each ring and of which at least one of the rings comprises one unsaturation; it being possible for the other ring to be saturated or aromatic.

A is preferably bicyclic, which means that at least two rings have two carbon atoms in common.

As examples of A, there may be mentioned, inter alia, norbornene and norbornadiene.

It is also possible to start with a substrate resulting from the linkage of a saturated or unsaturated aliphatic group and/or of a saturated, unsaturated or aromatic carbocycle.

There is the presence of at least one unsaturation in an aliphatic chain and/or in a ring.

In particular, the substrates of formula (I) are aimed at in which A represents a linear or branched, saturated or unsaturated aliphatic group bearing a cyclic substituent. The expression ring is preferably understood to mean a saturated, unsaturated or aromatic carbocyclic ring, preferably a cycloaliphatic or aromatic, in particular cycloaliphatic, ring comprising 6 carbon atoms in the ring or a benzene ring.

The acyclic aliphatic group may be linked to the ring by a valence bond, a heteroatom or a functional group and examples are given above.

A represents an aliphatic group bearing a cyclic substituent having at least one unsaturation in the aliphatic chain and/or in the ring.

In particular, the invention is aimed at the substrates consisting of an unsaturated aliphatic chain bearing a phenyl group and there may be mentioned in particular a styrenyl group.

In the case of a ring, the presence of substituents is not excluded insofar as they are compatible with the applicant envisaged. The substituents most often carried by the carbocycle are one or more alkyl groups, preferably three methyl groups, a methylene group (corresponding to an exocyclic bond), an alkenyl group, preferably an isopropenyl group.

As examples of aldehydes which can be used, there may be mentioned:

unsaturated aliphatic terpene aldehydes such as:
neral,
prenal,
geranial,
citronellal,
cyclocitral,
safranal,

unsaturated, monocyclic or polycyclic cycloaliphatic terpene aldehydes such as:
1,3,5-trimethyl-4-hydroxymethylcyclohexene,
campholenic aldehyde.

Among all the abovementioned aldehydes, the preferred aldehydes are the following:
citral,
prenal,
retinal,
cyclocitral,
safranal.

The compound to which the method according to the invention applies in a more particularly advantageous manner is the preparation of geranic acid.

The catalyst used in the method of the invention must work in a physical mode.

To this effect, the quantity of oxygen dissolved in the medium is limited by controlling various process parameters such as temperature, pressure and stirring. It is important that oxygen is consumed as soon as it arrives in the medium.

The catalyst used in the method of the invention is based on a metal called M₁ which is palladium, platinum or mixtures thereof.

There are preferably used platinum and/or palladium catalysts taken in all the available forms such as for example: platinum black, palladium black, platinum oxide, palladium oxide or the noble metal itself deposited on various supports such as carbon black, graphite, activated charcoal, activated aluminas and silicas or equivalent materials. Carbon-based catalytic masses are particularly suitable.

Generally, the metal is deposited in an amount of 0.5% to 95%, preferably of 1% to 5% of the weight of the catalyst.

The quantity of this catalyst to be used, expressed by weight of metal M₁ relative to that of the compound of formula (I), may vary from 0.001 to 10%, and preferably from 0.002 to 2%.

For more details on the catalysts, reference may be made to U.S. Pat. No. 3,673,257, FR-A-2 305 420 and FR-A-2 350 323.

According to a variant of the method of the invention, the metal M₁ is brought beforehand to the oxidation state zero by introducing formalin in any form (aqueous fomalin, trioxane or polyoxymethylene) in a suitable quantity. As a guide, it will be specified that the quantity to be used, expressed by weight of formalin per gram of metal, may vary from 0.02 g to 0.1 g/g.

Bismuth is used as activators. Preferably, bismuth is used in the form of free metals or of cations. In the latter case, the associated anion is not critical and it is possible to use any derivatives of these metals. Preferably, bismuth metal or its derivatives are used.

It is possible to use an inorganic or organic derivative of bismuth in which the bismuth atom is at an oxidation state greater than zero, for example equal to 2, 3, 4 or 5. The residue associated with bismuth is not critical as long as it satisfies this condition. The activator may be soluble or insoluble in the reaction medium.

Illustrative activator compounds which may be used in the method according to the present invention are: bismuth oxides; bismuth hydroxides; inorganic hydracid salts such as: bismuth chloride, bromide or iodide; inorganic oxyacid salts such as: bismuth sulfite, sulfate, nitrite, nitrate, phosphite, phosphate, pyrophosphate, carbonate or perchlorate.

Other appropriate compounds are also the salts of aliphatic or aromatic organic acids such as: bismuth acetate, propionate, benzoate, salicylate, oxalate, tartrate, lactate or citrate. These salts may also be bismuthyl salts.

By way of specific examples, there may be mentioned:

as oxides: BiO; Bi₂O₃: Bi₂O₄; Bi₂O₅,

as hydroxides: Bi(OH)₃,

as inorganic hydracid salts: bismuth chloride BiCl₃; bismuth bromide BiBr₃; bismuth iodide BiI₃,

as inorganic oxyacid salts: basic bismuth sulfite Bi₂(SO₃)₃, Bi₂O₃.5H₂O; neutral bismuth sulfate Bi₂(SO₄)₃; bismuthyl sulfate (BiO)HSO₄; bismuthyl nitrite (BiO)NO₂.0.5H₂O; neutral bismuth nitrate Bi(NO₃)₃.5H₂O; neutral bismuth phosphate BiPO₄; bismuth pyrophosphate Bi₄(P₂O₇)₃; bismuthyl carbonate (BiO)₂CO₃.0.5H₂O; neutral bismuth perchlorate Bi (ClO₄)₃.5H₂O; bismuthyl perchlorate (BiO)ClO₄,

as aliphatic or aromatic organic acid salts: bismuth acetate Bi (C₂H₃O₂)₃; bismuthyl propionate (BiO) C₃H₅O₂; basic bismuth benzoate C₆H₅CO₂Bi(OH)₂; bismuthyl salicylate C₆H₄CO₂(BiO) (OH); bismuth oxalate (C₂O₄)₃Bi₂; bismuth tartrate Bi₂(C₄H₄O₆)₃.6H₂O; bismuth lactate (C₆H₉O₅) OBi.7H₂O; bismuth citrate C₆H₅O₇Bi.

The bismuth derivatives which are preferably used to carry out the method according to the invention are: bismuth oxides; bismuth hydroxides; bismuth or bismuthyl salts of inorganic hydracids; bismuth or bismuthyl salts of inorganic oxyacids; bismuth or bismuthyl salts of aliphatic or aromatic organic acids.

A group of activators which are particularly suitable for carrying out the invention consists of: bismuth oxides Bi₂O₃ and Bi₂O₄; bismuth hydroxide Bi(OH)₃; neutral bismuth sulfate Bi₂(SO₄)₃; bismuth chloride BiCl₃; bismuth bromide BiBr₃; bismuth iodide BiL₃; neutral bismuth nitrate Bi(NO₃)₃.5H₂O; bismuthyl carbonate (BiO)₂CO₃.0.5H₂O; bismuth acetate Bi(C₂H₃O₂)₃; bismuthyl salicylate C₆H₄CO₂(BiO) (OH).

The quantity of activator used, expressed as the quantity of metal contained in the activator relative to the weight of the metal M₁ used, may vary widely. For example, this quantity may be as small as 1% and may be up to 200% of the weight of metal M₁ used and may even exceed it without any drawback. Advantageously, it is in the region of 100%.

The oxidation reaction carried out in accordance with the invention is performed in a basic medium.

As basic agents, use is made of alkali or alkaline-earth metal bases, among which there may be mentioned hydroxides such as sodium, potassium or lithium hydroxide.

For economic considerations, sodium or potassium hydroxide is used.

The concentration of the starting basic solution is not critical. The alkali metal hydroxide solution used has a concentration which is generally between 2 and 25%, preferably between 2 and 10% by weight.

The quantity of base introduced into the reaction medium is such that the ratio between the number of moles of OH— and the number of moles of aldehyde varies between 0.9 and 1.1, and is preferably equal to about 1.

If said compound has salifiable functional groups other than the carboxyl group formed, the quantity of base necessary to salify all the salifiable functional groups is then introduced.

The concentration by weight of the compound of formula (I) in the liquid phase is usually between 1% and 40%, preferably between 2% and 30%.

Water is present in the reaction medium whose quantity must be sufficient to solubilize the salt of the acid formed.

The water may be provided by the basic solution.

In accordance with the invention, the oxidation temperature is preferably chosen from a temperature range from 20° C. to 60° C., preferably between 30° C. and 40° C.

The procedure is generally carried out at atmospheric pressure, but it is possible to work at a pressure of between 1 and 20 bar.

As regards the stirring conditions, persons skilled in the art are capable of determining them in order to maintain a diffusion mode.

As a guide, it may be specified that in the case of a 3.2 liter reactor equipped with a stirrer of the inclined 4-blade type immersed in the reaction medium, the stirring conditions vary advantageously between 500 and 700 revolutions/min.

Practically, a way of carrying out the method consists in introducing the water, the basic agent, the catalyst based on palladium and/or platinum, the activator and then finally the aldehyde to be oxidized.

Next, the reaction medium, kept under an inert gas (for example nitrogen) stream, is heated to the desired reaction temperature and then oxygen or a gas containing it (air) is introduced.

The mixture is then stirred at the desired temperature until a quantity of oxygen corresponding to that necessary to convert the formyl group to a carboxyl group is consumed.

At the end of the reaction which preferably lasts for between 30 minutes and 6 hours, the carboxylic compound corresponding to formula (II) is recovered, which formula corresponding to formula (I); in which the CHO group is replaced by COOM; M representing the cation which corresponds to that of the base used.

Next, after cooling if necessary, the catalytic mass is separated from the reaction medium, for example by filtration.

In the next step, the resulting medium is acidified by adding a protonic acid of inorganic origin, preferably hydrochloric acid or sulfuric acid or of an organic acid such as for example methanesulfonic acid until there is obtained a pH less than the pKa of the acid obtained.

The acid concentration is unimportant and the commercially available forms are preferably used.

The acidification is generally performed between room temperature (most often between 15° C. and 25° C.).

The acid is then recovered conventionally according to conventional separation techniques, for example by distillation. The organic phase may be extracted with a solvent, for example an aromatic hydrocarbon, preferably toluene and then the organic phase is distilled off in order to recover first the solvent, possibly the starting aldehyde, and then the carboxylic acid formed and possibly by-products formed.

It corresponds to formula (III), which formula corresponds to formula (I), in which the CHO group is replaced by COOH.

The method of the invention applies particularly to the preparation of unsaturated carboxylic acids of the terpene type and more preferably to geranic acid.

It is possible to carry out the reaction in any type of reactor provided that the process parameters are chosen which make it possible to work in physical mode in relation to the oxygen.

Various exemplary embodiments of the invention are given by way of illustration.

In the examples, the rate of conversion (RC) corresponds to the ratio between the number of substrates converted and the number of moles of substrate used.

The yield (actual yield) corresponds to the ratio between the number of moles of product formed (carboxylic acid) and the number of moles of substrate used.

### EXAMPLE 1

100 g of water, 17.5 g of an aqueous sodium hydroxide solution at 30% by weight, 12 g of platinum on activated charcoal containing 50% by weight of water and having a titer of 2.5% by weight of platinum expressed relative to the dry catalyst (platinum+support), 0.167 g of bismuth oxide and 0.5 g of an aqueous solution of formalin at 5% by weight are introduced into a 150 ml glass reactor having a diameter of 80 mm, equipped with a stirring system (central and counter-blade stirring).

The stirring is performed using a stirrer of the inclined 4-blade type for which the position of the helix relative to the height of the liquid in the reactor is at one third relative to the bottom of the reactor.

Stirring is carried out for 30 min at room temperature under a nitrogen stream.

20 g of citral are then introduced and air is introduced at a flow rate of 35 ml/minute and the medium is heated at 45° C. at a stirring rate of 600 rpm.

The reaction is performed at atmospheric pressure.

The reaction should be performed in diffusion mode.

After 4 hours under these conditions, the reaction medium is analyzed by gas chromatography (GC) and the following results are obtained:

rate of conversion=71%

yield of geranic acid (mixture of E and Z isomers)=21%

### EXAMPLE 2

100 g of water, 17.5 g of an aqueous sodium hydroxide solution at 30% by weight, 12 g of platinum on activated charcoal containing 50% by weight of water and having a titer of 2.5% by weight of platinum expressed relative to the dry catalyst, 0.84 g of bismuth oxide and 0.5 g of an aqueous solution of formalin at 5% by weight are introduced into a reactor as described in Example 1.

Stirring is carried out for 30 min at room temperature under a nitrogen stream.

20 g of citral are then added and air under a stream of 35 ml/min while maintaining the temperature at 45° C.

After 4 hours under these conditions, the reaction medium is analyzed by GC and the following results are obtained:

rate of conversion=60%

yield=15%

### EXAMPLE 3

100 g of water, 17.5 g of an aqueous sodium hydroxide solution at 30% by weight, 12 g of platinum on activated charcoal containing 50% by weight of water and having a titer of 2.5% by weight of platinum expressed relative to the dry catalyst, 0.167 g of bismuth oxide and 0.5 g of an aqueous solution of formalin at 5% by weight are introduced into a reactor as described in Example 1.

Stirring is carried out for 30 min at room temperature under a nitrogen stream.

20 g of citral are then introduced and air is introduced at a flowrate of 35 ml/minute and the medium is heated to 35° C.

After 4 hours under these conditions, the reaction medium is analyzed by GC and the following results are obtained:

rate of conversion=50%

yield=30%

### EXAMPLE 4

100 g of water, 17.5 g of an aqueous sodium hydroxide solution at 30% by weight, 10.5 g of palladium on activated charcoal containing 50% by weight of water and having a titer of 3.0% by weight of palladium expressed relative to the dry catalyst, 0.167 g of bismuth oxide and 0.5 g of an aqueous solution of formalin at 5% by weight are introduced into a reactor as described in Example 1.

Stirring is carried out for 30 min at room temperature under a nitrogen stream.

20 g of citral are then introduced and air is introduced at a flowrate of 35 ml/minute and the medium is heated to 45° C.

After 4 hours under these conditions, the reaction medium is analyzed by GC and the following results are obtained:

rate of conversion=36%

yield=9%

### EXAMPLE 5

100 g of water, 17.5 g of an aqueous sodium hydroxide solution at 30% by weight, 3.0 g of platinum on dry activated charcoal and having a titer of 3.5% by weight of platinum plus 3.5% by weight of Bi, are introduced into a reactor as described in Example 1, 0.5 g of an aqueous solution of formalin at 5% by weight is added.

Stirring is carried out for 30 min at room temperature under a nitrogen stream.

20 g of citral are then introduced and air is introduced at a flowrate of 60 ml/minute and the medium is heated to 45° C.

After 4 hours under these conditions, the reaction medium is analyzed by GC and the following results are obtained:

rate of conversion=31%

yield=18%

### COMPARATIVE EXAMPLE 6

In this example, there is no bismuth.

100 g of water, 17.5 g of an aqueous sodium hydroxide solution at 30% by weight, 3.0 g of dry platinum on activated charcoal and having a titer of 3.5% by weight of platinum are introduced into a 150 ml glass reactor, 0.5 g of an aqueous solution of formalin at 5% by weight is added.

Stirring is carried out for 30 min at room temperature under a nitrogen stream.

20 g of citral are then introduced and air is introduced at a flowrate of 35 ml/minute and the medium is heated to 45° C.

After 4 hours under these conditions, the reaction medium is analyzed by GC.

The results obtained are the following:

rate of conversion=24%

yield=1%

### COMPARATIVE EXAMPLE 7

50 g of water, 50 ml of dioxane, 17.5 g of an aqueous sodium hydroxide solution at 30% by weight, 12 g of platinum on activated charcoal containing 50% of water and having a titer of 2.5% by weight of platinum expressed relative to the dry catalyst, 0.167 g of bismuth oxide and 0.5 g of an aqueous solution of formalin at 5% by weight are introduced into a reactor as described in Example 1.

Stirring is carried out for 30 min at room temperature under a nitrogen stream.

20 g of citral are then introduced and air is introduced at a flowrate of 35 ml/minute and the medium is heated to 45° C.

After 4 hours under these conditions, the reaction medium is analyzed by GC and the following results are obtained:

rate of conversion=81%

yield=21%

In a monophasic medium, the citral undergoes degradation of 6-methyl-1-hepten-2-one.

1-27. (canceled)

28. A method for preparing an unsaturated carboxylic acid from the corresponding aldehyde, comprising the steps of:
a) oxidizing said aldehyde, in a controlled basic medium and using molecular oxygen or a gas containing it, in the presence of a catalyst based on palladium and/or platinum and of an activator based on bismuth, under conditions such that the oxidation occurs in a diffusion mode, and
b) recovering the carboxylic acid formrd in step a).

29. The method as claimed in claim 28, wherein the starting aldehyde is an aliphatic or cycloaliphatic aldehyde having at least one unsaturation, a double bond or a triple bond.

30. The method as claimed in claim 29, wherein the starting aldehyde is an aliphatic aldehyde having two double bonds of which at least one is conjugated with the carbonyl group.

31. The method as claimed in claim 30, wherein the starting aldehyde is a terpene aldehyde.

32. The method as claimed in claim 28, wherein the starting aldehyde corresponds to formula (I):
A-CHO (I)
wherein:
A represents a hydrocarbon group having at least one unsaturation having from 4 to 19 carbon atoms which is a linear or branched, saturated or unsaturated acyclic aliphatic group; a monocyclic or polycyclic, saturated or unsaturated or aromatic carbocyclic group; a linkage of a saturated or unsaturated aliphatic group and/or of a saturated, unsaturated or aromatic carbocycle.

33. The method as claimed in claim 32, wherein A represents a linear or branched acyclic aliphatic group having from 4 to 19 carbon atoms having one or more unsaturations in the chain, which are single or conjugated double bonds or triple bonds, said unsaturation being at the chain end and/or alternatively inside the chain and/or conjugated with the CO group.

34. The method as claimed in claim 33, wherein A represents a linear or branched alkyl group having from 4 to 19 carbon atoms and having at least one double bond, optionally two double bonds of which at least one is conjugated with the CO group.

35. The method as claimed in claim 32, wherein A represents a carbocycle having from 3 to 8 carbon atoms in the ring, optionally 5 or 6 and having 1 or 2 unsaturations in the ring, optionally 1 or 2 double bonds.

36. The method as claimed in claim 35, wherein A represents a cycloalkyl group having 5 or 6 carbon atoms and having a double bond.

37. The method as claimed in claim 32, wherein A represents a polycyclic carbocyclic group having from 3 to 6 carbon atoms in each ring and of which at least one of the rings presents one unsaturation, optionally the other ring being saturated or aromatic.

38. The method as claimed in claim 28, wherein the starting aldehyde is citral, prenal, retinal, cyclocitral, or safranal.

39. The method as claimed in claim 28, wherein the platinum and/or palladium catalyst is provided in the form of platinum black, palladium black, platinum oxide, palladium oxide or the noble metal itself deposited on a support.

40. The method as claimed in claim 28, wherein the catalyst is used in a quantity, expressed by weight of metal M₁ relative to that of the compound of formula (I), of from 0.001 to 10%.

41. The method as claimed in claim 28, wherein the activator is an organic or inorganic derivative of bismuth chosen from the group consisting of:
bismuth oxides; bismuth hydroxides; bismuth salts of inorganic hydracids;
bismuthyl salts of inorganic hydracids; bismuth salts of inorganic oxyacids, bismuthyl salts of inorganic oxyacids; bismuth salts of aliphatic or aromatic organic acids; and bismuthyl salts of aliphatic or aromatic organic acids.

42. The method as claimed in claim 41, wherein the bismuth derivative is chosen from the group consisting of: bismuth oxides Bi₂O₃ and Bi₂O₄; bismuth hydroxide Bi(OH)₃; bismuth chloride BiCl₃; bismuth bromide BiBr₃; bismuth iodide BiL₃; neutral bismueth sulfate Bi₂(SO₄)₃; neutral bismuth nitrate Bi(NO₃)₃.5H₂O; bismuthyl carbonate (BiO)₂CO₃.0.5H₂O; bismuth acetate Bi(C₂H₃O₂)₃: and bismuthyl salicylate C₆H₄CO₂(BiO)OH.

43. The method as claimed in claim 42, wherein the activator is in a quantity expressed by weight of bismuth relative to the weight of the metal M₁ of between 1 and 200%.

44. The method as claimed in claim 28, wherein the basic agent is sodium hydroxide or potassium hydroxide.

45. The method as claimed in claim 28, wherein the quantity of base added into the reaction medium is such the number of moles of OH— and the number of moles of aldehyde are in a ratio of between 0.9 and 1.1.

46. The method as claimed in claim 28, wherein water is present in the reaction medium in a quantity sufficient to solubilize the salt of the acid formed.

47. The method as claimed in claim 28, wherein the oxidation takes place at a temperature of between 20° C. and 60° C.

48. The method as claimed in claim 28, being carried out at atmospheric pressure.

49. The method as claimed in claim 28, being carried out under stirring conditions which are such that the reaction mode is a diffusion mode.

50. The method as claimed in claim 46, wherein the water, the basic agent, the catalyst based on palladium and/or platinum, the activator, and then finally the aldehyde to be oxidized, are being added.

51. The method as claimed in claim 50, wherein the metal M₁ is reduced with formalin.

52. The method as claimed in claim 50, wherein the reaction mixture maintained under a stream of inert gas is heated to the desired reaction temperature and then oxygen or a gas containing it is introduced.

53. The method as claimed in claim 52, wherein the medium is stirred at the desired temperature until a quantity of oxygen corresponding to that necessary to convert the formyl group to a carboxyl group is consumed.

54. The method as claimed in claim 28, wherein in step b) the carboxylic acid formed a) is recovered after an acid treatment.
